SEQUENCE LISTING (8 sequences)

SEQ ID NO: 1 - DNA, 19 bp, Artificial Sequence
  Synthetic
aaaaaaaaatgaactaacg

SEQ ID NO: 2 - DNA, 19 bp, Artificial Sequence
  Synthetic
aaaaaaaaatgaactaacg

SEQ ID NO: 3 - DNA, 20 bp, Artificial Sequence
  Synthetic
aaaaaaaaaacgttagttca

SEQ ID NO: 4 - DNA, 37 bp, Artificial Sequence
  Synthetic
caatcatgagcagcctagcagagaagtaaaaaaaaaa

SEQ ID NO: 5 - DNA, 37 bp, Artificial Sequence
  Synthetic
acttctctgctaggctgctcatgattgcgttagttca

SEQ ID NO: 6 - DNA, 37 bp, Artificial Sequence
  Synthetic
acttctctgctaggctgctcatgattgcgttaattca

SEQ ID NO: 7 - DNA, 37 bp, Artificial Sequence
  Synthetic
acttctctgctaggctgctcatgattgcgttacttca

SEQ ID NO: 8 - DNA, 37 bp, Artificial Sequence
  Synthetic
acttctctgctaggctgctcatgattgcgttatttca

(19) United States
(12) Patent Application Publication
(10) Pub. No.: US 2013/0143331 A1
(43) Pub. Date: Jun. 6, 2013

(54) COMPOSITIONS AND METHODS FOR PHOTOCONTROLLED HYBRIDIZATION AND DEHYBRIDIZATION OF A NUCLEIC ACID

(71) Applicants: David Ginger, Seattle, WA (US); Yunqi Yan, Seattle, WA (US); Jennifer Chen, Toronto (CA)

(72) Inventors: David Ginger, Seattle, WA (US); Yunqi Yan, Seattle, WA (US); Jennifer Chen, Toronto (CA)

(21) Appl. No.: 13/681,209

(22) Filed: Nov. 19, 2012

Related U.S. Application Data
(60) Provisional application No. 61/561,372, filed on Nov. 18, 2011.

(51) Int. Cl. C12Q 1/68

(52) U.S. Cl. CPC C12Q 1/6834
USPC 436/174; 536/24.3; 428/402

(57) ABSTRACT

Compositions and methods are provided that enable light-controlled hybridization between two nucleic acid sequences.

### CROSS-REFERENCE TO RELATED APPLICATION

This application claims the benefit of U.S. Patent Application No. 61/561,372, filed Nov. 18, 2011, the disclosure of which is hereby incorporated by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 40300_Seq_Final_{—}2012-11-19.txt. The text file is 2.55 KB; was created on Nov. 19, 2012; and is being submitted via EFS-Web with the filing of the specification.

### FEDERAL FUNDING STATEMENT

This invention was made with government support under contract FA9550-10-1-0474, awarded by the Air Force Office of Scientific Research, and contract CMMI-0709131, awarded by the National Science Foundation. The government has certain rights in the invention.

### BACKGROUND

Hydrogen bonding between complementary bases in DNA leads to the hybridization of two strands into a duplex structure. Conventionally, thermal energy such as heat, or changes in ionic strength (salt gradients), are required to melt (dehybridize) the two strands when performing analytical techniques, such as hybridization stringency washes. However, temperature and concentration gradients can be difficult to control precisely in the context of such automated solution-based assays, which may hinder precision. Therefore, alternative means for dehybridizing nucleic acids in solution would be desirable so as to improve present solution-based assays and possibly enable new techniques, as well.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a composition is provided. In one embodiment, the composition includes:

a surface;

a first nucleic acid sequence attached to the surface; and

a photoswitchable molecule incorporated into the first nucleic acid sequence; wherein the photoswitchable molecule is capable of undergoing a structural change from a first conformation to a second conformation upon illumination by a first wavelength of light, wherein the structural change alters a hybridization property of the first nucleic acid sequence in relation to a second nucleic acid sequence.

In another aspect, a method of altering a hybridization property between a first nucleic acid sequence and a second nucleic acid sequence is provided. In one embodiment, the method includes the steps of:

(a) providing a solution, comprising:
(i) a first nucleic acid sequence attached to a surface; and
(ii) a second nucleic acid sequence that is not attached to the surface;
   wherein at least one of the first nucleic acid sequence and the second nucleic acid sequence incorporates a photoswitchable molecule; wherein the photoswitchable molecule is capable of undergoing a structural change from a first conformation to a second conformation upon illumination by a first wavelength of light, wherein the structural change alters a hybridization property of the first nucleic acid sequence in relation to the second nucleic acid sequence; and

(b) altering the hybridization property by illuminating the photoswitchable molecule with the first wavelength of light.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A. Photoisomerization of an example photoswitch, azobenzene, from trans to cis with UV light and cis to trans with blue light (top). When azobenzene is incorporated into DNA, photoregulation of duplex hybridization can be achieved (bottom).

FIG. 1B. Photocontrolled aggregation and disaggregation of gold nanoparticles functionalized with azobenzene modified DNA. Upon UV irradiation, aggregates disassemble to yield single nanoparticles.

FIG. 2. Photoswitch-modified DNA-functionalized gold nanoparticle conjugates. Gold nanoparticles are functionalized with azobenzene-modified, thiol-terminated DNA. Hybridization of nanoparticles bearing complementary sequences is then controllable by illumination with UV and blue light via trans-cis photoisomerization of azobenzene.

FIG. 3. DNA Sequences and 3-strand capture motif used in the photostringency experiments. DNA functionalized gold nanoparticles (Box A) form aggregates in the presence of target nucleic acids (Box B), which dissociate at different rates upon UV irradiation (Box C) depending on the presence or absence of a single-base mismatch in the target.

FIGS. 4A-4C. Reversible photo-controlled assembly and disassembly of DNA-nanoparticle conjugates made with azobenzene-modified oligonucleotides. (4A) Photographs of the solution containing [SEQ ID NO:1]-AuNPs and [SEQ ID NO:3]-AuNPs after alternating UV and blue irradiation. (4B) Corresponding UV-Vis spectra of the solution in 4A for three photoswitching cycles, with the extinction at 526 nm after each irradiation plotted in (4C).

FIGS. 5A-C. Aggregation of [SEQ ID NO:1]-AuNPs and [SEQ ID NO:3]-AuNPs as monitored by UV-vis extinction at 526 nm for different pre-mixing illumination conditions on [SEQ ID NO:1]-AuNPs. (5A) Illumination with UV before mixing (circles) prevents hybridization and nanoparticle aggregation, while nanoparticles not exposed to UV (diamonds) or to UV then blue light (triangles) both form aggregates. (5B) Extinction spectra of control nanoparticle assemblies linked by native DNA (without azobenzene) upon UV and subsequent blue illumination. (5C) Spectral evolution of [SEQ ID NO:1]-AuNP and [SEQ ID NO:3]-AuNP assemblies at different UV time intervals.

FIGS. 6A-D. (6A) Calibration curve showing linear dependence of scattering intensity on cross sectional area of the aggregates obtained from SEM-darkfield correlation. The linear fit is y=−11.2±10.7+0.0070±0.0005x, with a Pearson's correlation of 0.89. The inset shows a SEM image of a typical surface-anchored multi-nanoparticle assembly. Normalized scattering intensity obtained from an average of 30-40 aggregates as a function of UV irradiation time at: (6B) different temperatures; and (6C) different light intensities. (6D) The same curves in (6C) plotted as a function of photon dose.

FIGS. 7A-7C. Photostringency experiments demonstrating the discrimination of complementary from single-base-mismatched sequences linking [SEQ ID NO:1]-AuNP and [SEQ ID NO:4]-AuNP assemblies. (7A) LSPR peak wavelengths and (7B) changes in the extinction at 526 nm of the four solutions as a function of UV photon dose. The complementary sequence (squares) is clearly distinguished from the single-base mismatches. (7C) Photographs of the solutions before and after 1.6 J (2 h) of UV irradiation exposure.

FIG. 8. Reversible photoswitching of [SEQ ID NO:1]-AuNPs and [SEQ ID NO:3]-AuNPs for 10 cycles shown by the LSPR peak shift of the solution.

FIG. 9. Schematic showing a representative photoswitching apparatus.

FIG. 10. Melting curves of AuNP assemblies linked by azobenzene-modified DNA ([SEQ ID NO:1]+[SEQ ID NO:3]) or normal DNA ([SEQ ID NO:2]+[SEQ ID NO:3]) in 0.01 M PBS, 0.1 M NaCl and 0.01% SDS. Both assemblies show sharp melting transitions, with a higher melting temperature observed for azobenzene-modified DNA-AuNP assemblies.

FIGS. 11A-C. (11A) Darkfield images of aggregates on a substrate at different duration of UV irradiation (with intermittent exposure to darkfield light when images were captured). (11B) Darkfield image at low magnification showing an area that had been UV irradiated vs. an area without irradiation at same temperature. (11C) Schematic showing the microscope setup.

FIG. 12. Reversible photoswitching of a 3-strand motif: [SEQ ID NO:1]-AuNPs and [SEQ ID NO:4]-AuNPs linked by [SEQ ID NO:5] (at 0.01 M phosphate buffer, 0.05 M NaCl, 45° C.). Initially the solution has a LSPR peak at 571 nm, which then cycles between 526 nm and 534 nm upon alternating UV and blue irradiation for 0.5 h.

### DETAILED DESCRIPTION

Compositions and methods are provided that enable light-controlled hybridization between two nucleic acid sequences.

In one aspect, a composition is provided. In one embodiment, the composition includes:

a surface;

a first nucleic acid sequence attached to the surface; and

a photoswitchable molecule incorporated into the first nucleic acid sequence; wherein the photoswitchable molecule is capable of undergoing a structural change from a first conformation to a second conformation upon illumination by a first wavelength of light, wherein the structural change alters a hybridization property of the first nucleic acid sequence in relation to a second nucleic acid sequence.

Specifically, the composition incorporates the photoswitchable molecule into the structure of the first nucleic acid sequence. The photoactive properties of the modified first nucleic acid are then utilized to control the hybridization and/or dehybridization of the first nucleic acid sequence with the second nucleic acid sequence.

As used herein, the term “nucleic acid” refers to DNA (deoxyribonucleic acid) or RNA (ribonucleic acid), and variants thereof. Nucleic acids are synonymous with polynucleotides. Furthermore, the term “nucleic acid sequence” refers to a sequence (i.e., a plurality) of adjacent nucleotides, which may constitute an entire nucleic acid or a portion thereof. The nucleic acid sequences referred to herein can be sequences on separate nucleic acid chains (e.g., one sequence on each strand of double-stranded DNA) or on a single nucleic acid chain (e.g., RNA that is folded over onto itself so as to arrange the two different sequences in close proximity).

A “photoswitchable” molecule is one that changes conformations when illuminated with electromagnetic radiation (“light”). In certain embodiments, the photoswitchable molecule photoisomerizes from cis to trans or vice versa. In certain embodiments, the photoswitchable molecule is reversibly photoswitchable, such that one wavelength of light changes the conformation of the molecule from a first state to a second state; and a second wavelength of light reverses the conformation change from the second state back to the first state. A representative photoswitchable molecule is azobenzene (and photoswitchable analogs thereof). Further representative photoswitchable molecules include other azobenzenes, stilbenes, spiropyrans, fulgides, diarylethenes, diphenylpolyenes, dihydro-indolizines, diarylethanes, chromenes, napthopyrans, spiropyrans, fulgides, fulgimides, spiroxazines, and other compounds undergoing reversible structural changes upon photoexcitation.

In one embodiment, the photoswitchable molecule is incorporated into the first nucleic acid sequence (to provide a “photoswitchable” nucleic acid. Such incorporation may be by intercalation and/or covalent bond between the photoswitchable molecule and the first nucleic acid sequence (e.g., bound via a base). For instance, azobenzene (as used in exemplary embodiments herein) are linked by a covalent bond to the nucleic acid sequence, but inserted by intercalation. It would be possible for the photoswitchable molecule to be linked by a covalent bond at a site on a nucleic acid sequence that does not allow it to intercalate. Finally, it is possible in some instances to have a photoswitchable molecule that intercalates but is not linked by a covalent bond.

In another embodiment, the photoswitchable molecule is incorporated into the second nucleic acid sequence (e.g., the sequence not attached to the surface). In yet another embodiment, there are photoswitchable molecules incorporated into both the first nucleic acid sequence and the second nucleic acid sequence; these photoswitchable molecules may be the same or different on each nucleic acid sequence.

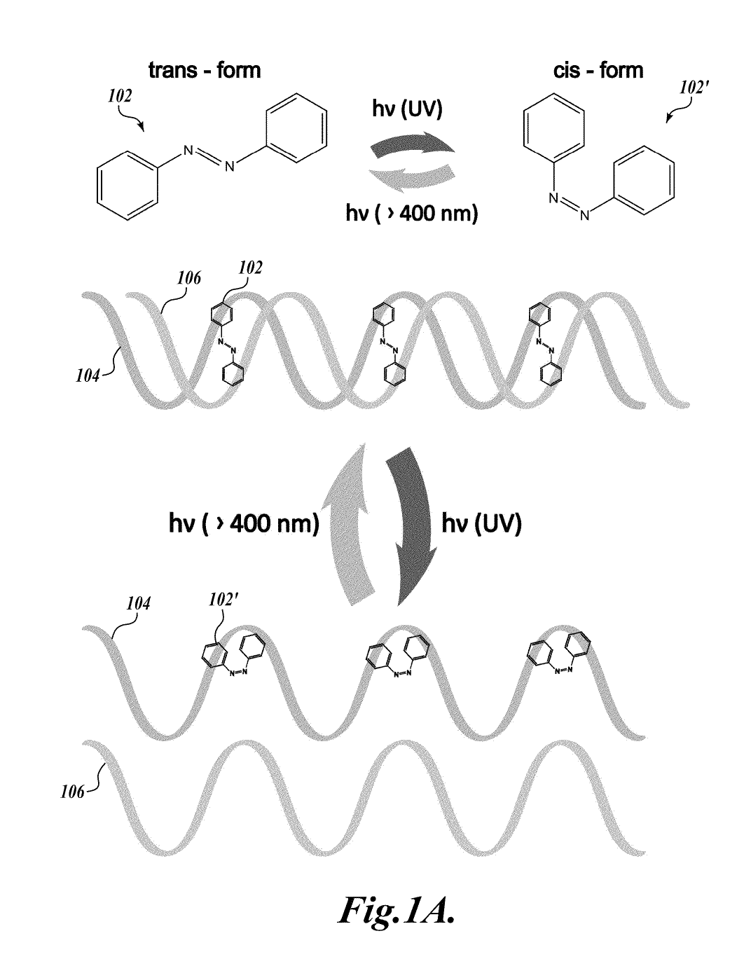

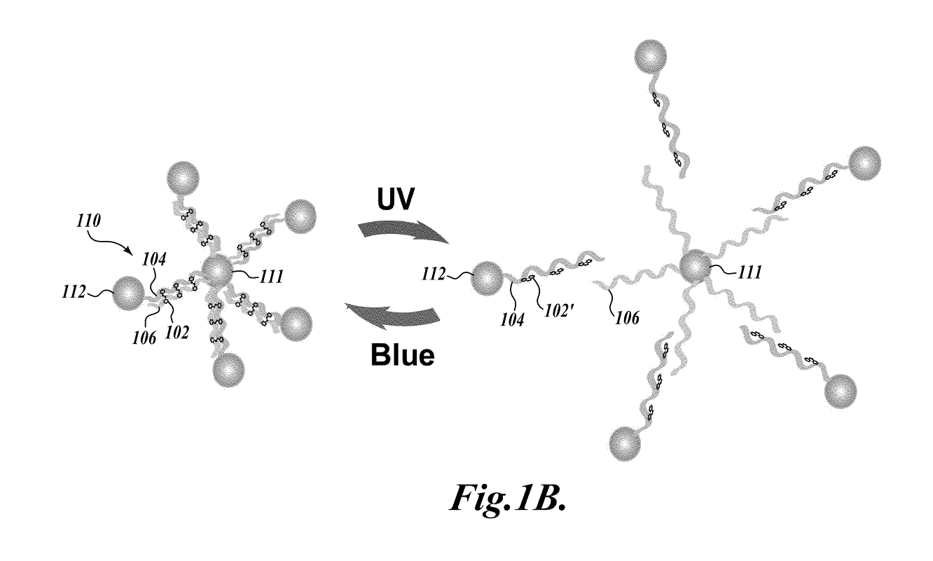

As an illustrative example of photocontrolled hybridization, FIG. 1A illustrates duplex DNA consisting of a first nucleic acid sequence 104, incorporating azobenzene molecules 102, and a second nucleic acid sequence 106. In the initial state, azobenzene 102 exists in the (lower energy) trans form and the DNA 104 and 106 forms a stable duplex structure in which the azobenzene 102 molecules intercalates between the DNA bases via π-π stacked interaction. For efficient photoregulation, multiple azobenzene 102 molecules spaced two nucleotides apart are incorporated in one strand of DNA 104. In this example, the second strand 106 includes native nucleotides without modifications. Upon UV irradiation (at a first wavelength of light), trans-azobenzene 102 photoisomerizes to the (higher energy) cis-azobenzene 102′, which leads to dehybridization of the duplex. The duplex dehybridizes because of changes in the structural conformation of the system induced by the azobenzene and the decrease in the overall energetic stability of the duplex. The reverse isomerization occurs with blue light irradiation (at a second wavelength, greater than the first wavelength); and subsequent cycling of DNA hybridization can be carried out by alternating the light source wavelength.

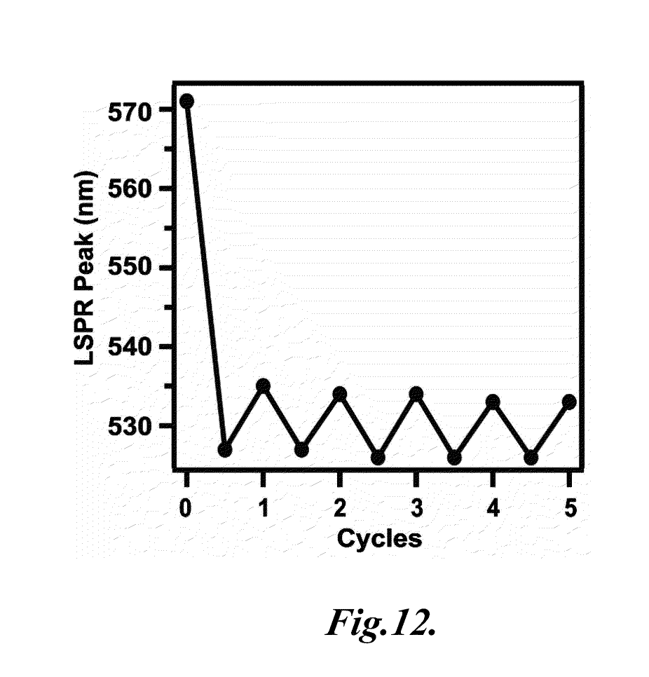

In one embodiment, the structural change is reversible upon illumination by a second wavelength of light that is different than the first wavelength of light. For example, after UV irradiation, cis-azobenzene can be photoisomerized back to the trans form by irradiating with blue light, thereby inducing re-aggregation of the nanoparticles. FIG. 12 shows the cycling of the plasmonic properties of the solution when the linker analyte is present, where SPR peak shifts between 526 nm and 534 nm after UV and blue irradiation, respectively. The solution can be cycled many times without noticeable deterioration of the optical properties, suggesting good photostability. This target-induced light modulated optical signal is unique to the disclosed systems and can be used to distinguish target binding from any isotropic background noise.

In one embodiment, the first wavelength and the second wavelength are independently selected from the group consisting of near-infrared, visible, and ultraviolet wavelengths.

In one embodiment, the first wavelength is less than the second wavelength and the hybridization property is altered to stabilize hybridization of the first nucleic acid sequence with the second nucleic acid sequence.

In one embodiment, the first wavelength is less than the second wavelength and the hybridization property is altered to destabilize hybridization of the first nucleic acid sequence with the second nucleic acid sequence more likely.

In one embodiment, the first wavelength is greater than the second wavelength and the hybridization property is altered to destabilize hybridization of the first nucleic acid sequence with the second nucleic acid sequence more likely.

In one embodiment, the first wavelength is greater than the second wavelength and the hybridization property is altered to stabilize hybridization of the first nucleic acid sequence with the second nucleic acid sequence.

As used herein, the term “hybridization property” refers to any characteristic that affects the ability of the first nucleic acid sequence to hybridize with the second nucleic acid sequence. At the extremes, the hybridization property is altered by the photoswitchable molecule to either entirely hybridize the two nucleic acid sequences (i.e., bind them together) or entirely dehybridize the two nucleic acid sequences (i.e., remove all binding forces between them). However, in certain embodiments, the photoswitchable molecule only acts to stabilize (i.e., make binding more energetically or entropically favorable) or destabilize (i.e., make binding less energetically or entropically favorable) the hybridization between the nucleic acid sequences.

In situations where the photoswitchable molecule does not completely hybridize or dehybridize the two nucleic acid sequences, other mechanisms can be used to complete the hybridization or dehybridization. For example, as will be discussed further in the EXAMPLE, a photoswitchable molecule can be used to destabilize hybridization between two bound nucleic acids, without actually dehybridizing them completely. This destabilization manifests itself when the temperature of the bound nucleic acids is raised:

the destabilized nucleic acids have a lowered melting temperature (i.e., dehybridization temperature) than if the photoswitchable molecule was not used to destabilize hybridization. Therefore, in certain embodiments, the photoswitchable molecule only contributes to stabilization and/or destabilization of hybridization, with other mechanisms, such as temperature and/or ion concentration, completing the hybridization/dehybridization. Conversely, in one embodiment, the temperature is kept constant during hybridization/dehybridization. In another embodiment, ion concentration temperature is kept constant during hybridization/dehybridization.

At least the first nucleic acid is attached to the surface. As used herein, “attached” means bound to or otherwise immobilized on the surface. This attachment may be covalent, ionic, electrostatic, or any other mechanism know to those of skill in the art. The first nucleic acid sequence can be directly attached to the surface or can be attached to the surface via a linker (e.g., a different portion of the nucleic acid strand).

The surface acts as an attachment point for one or more nucleic acids. At least the first nucleic acid sequence is attached to the surface. In certain embodiments a plurality of nucleic acid sequences (and/or strands) are attached to the surface.

In one embodiment, the surface is a surface of a core, wherein a core is defined as a particle of micro- or nano-scale size, depending on various factors described below. In other embodiments, the surface is a planar surface, such as can be found on an assay chip. Such assay chips are well known to those of skill in the art.

In certain embodiments the core functions as a reporting structure that can be detected. For example, in certain embodiments, the core is a nano-scale gold particle that exhibits surface plasmon resonance (SPR) such that optical absorbance spectroscopy (e.g., UV-vis) can be used to detect the core in solution. While optical detection schemes are primarily described herein, it will be appreciated that any detection scheme known to those of skill in the art can be used, including electrical or magnetic detection techniques. The composition of the core can be modified as necessary to facilitate the detection technique.

In certain embodiments, the core is a material that has an SPR. When photoswitchable nucleic acids are combined with plasmon-resonant metal nanoparticles, photoswitchable optical properties are created. The photoswitchable optical properties arise from the changes in the plasmon coupling of nanoparticles due to photocontrolled hybridization and dehybridization of the nucleic acids.

In certain embodiments, the core has an external surface that is a metal. Exemplary metals include gold, silver, aluminum, and combinations thereof, including alloys and core/shell particles. In these embodiments, the entire particle may be the single SPR metal, or the SPR metal may only coat a non-SPR metal core, such as a semiconductor or an insulator, as long as the particle as a whole has an SPR or is otherwise detectable as desired. Additional possible core materials include silicon, CdSe, CdS, ZnS, ZnO, polystyrene, latex, Fe₂O₃, CdSe/ZnS core/shell structures, copper, cobalt, platinum, and their respective oxides, and chalcogenides.

In other embodiments, the core is an insulator or semiconductor that does not have an SPR but is useful in an alternative detection scheme, such as by non-SPR optical detection or electrical detection.

In one embodiment, the shape of the core is selected from a sphere, a cylinder, an ellipsoid, a polyhedron, a prism, a rod, and a wire. The shape of the core may contribute to the detection properties, as will be appreciated by those of skill in the art (e.g., nano-rods may have different optical properties than nano-spheres).

In one embodiment, the core has a critical dimension of from 1 nm to 200 nm. The nano-scale size is critical particularly for optical detection techniques (e.g., SPR detection) and to facilitate the reversible aggregation/disaggregation of multiple cores together in a solution (e.g., because larger cores tend to aggregate/adhere to surfaces without complementary DNA).

In another embodiment, the core has a critical dimension of greater than one micron. In certain embodiments, such micron-sized cores are formed from polymer or silica.

In one embodiment the core is optically detectable by changes in absorption, light scattering, or photoluminescence that are triggered by changes in the hybridization state of the first nucleic acid sequence in relation to the second nucleic acid sequence. Furthermore, it is contemplated that the photoswitchable optical properties can be detected by many methods, such as a UV-Vis spectrophotometer, visually as a color change by naked eye in bulk solution, monitored at the single nanostructure level using dark-field microscope coupled with a fiber optic spectrometer, or detected by silver amplification on a chip.

On a single nanostructure level, the linked nanoparticles disaggregate within tens of seconds to minutes depending on the temperature and light intensity applied.

In another aspect, a method of altering a hybridization property between a first nucleic acid sequence and a second nucleic acid sequence is provided. In one embodiment, the method includes the steps of:

(a) providing a solution, comprising:
(i) a first nucleic acid sequence attached to a surface; and
(ii) a second nucleic acid sequence that is not attached to the surface; wherein at least one of the first nucleic acid sequence and the second nucleic acid sequence incorporates a photoswitchable molecule; wherein the photoswitchable molecule is capable of undergoing a structural change from a first conformation to a second conformation upon illumination by a first wavelength of light, wherein the structural change alters a hybridization property of the first nucleic acid sequence in relation to the second nucleic acid sequence; and

(b) altering the hybridization property by illuminating the photoswitchable molecule with the first wavelength of light.

In one embodiment, the surface is a surface of a core, wherein a core is defined as a particle of micro- or nano-scale size, depending on various factors described below. In other embodiments, the surface is a planar surface, such as can be found on an assay chip.

In one embodiment, the photoswitchable molecule is incorporated into the first nucleic acid sequence. In another embodiment, the photoswitchable molecule is incorporated into the second nucleic acid sequence.

In one embodiment, the first nucleic acid sequence and the second nucleic acid sequence are complementary.

In one embodiment, the first nucleic acid sequence and the second nucleic acid sequence are partially complementary and partially non-complementary when hybridized. The partially non-complementary aspect can be a nucleic acid mismatch, an abasic site, a modified base, or a combination thereof. In a further embodiment, the structural change destabilizes hybridization of the first nucleic acid sequence with the second nucleic acid sequence, and wherein said destabilized hybridization requires a first amount of photonic energy that is less than a second amount of photonic energy as defined by the amount of photonic energy required to destabilized hybridization of the first nucleic acid with the second nucleic acid to the same extent if they were more complementary. That is to say that the greater the extent of mismatch, the less photonic energy required to destabilize hybridization. As used herein, the term “photonic energy” refers to the amount of electromagnetic energy absorbed by the photoswitchable molecule. This is sometimes referred to as “photon dose.”

Because photonic energy can be controlled based on the wavelength and power of the light source, as well as the exposure time, the provided system and methods affords great control over when a photoswitch occurs, and to what extent it occurs. In this regard, if many photoswitchable molecules are incorporated into a nucleic acid, the switching light can be configured to either provide sufficient energy to the system so as to instantly switch all of the photoswitchable molecules, or the switching energy may be delivered more slowly, such that the photoswitchable molecules switch over the course of an elongated timeframe.

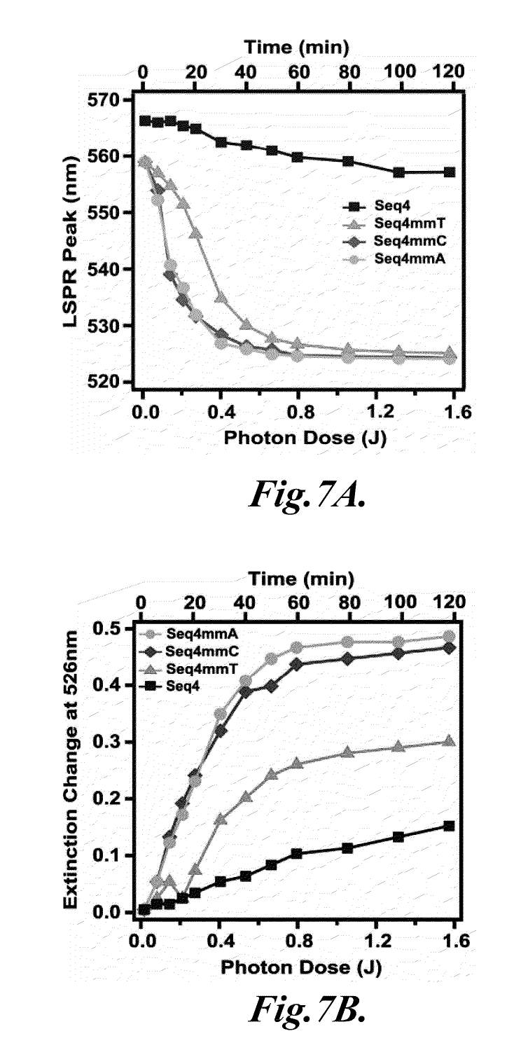

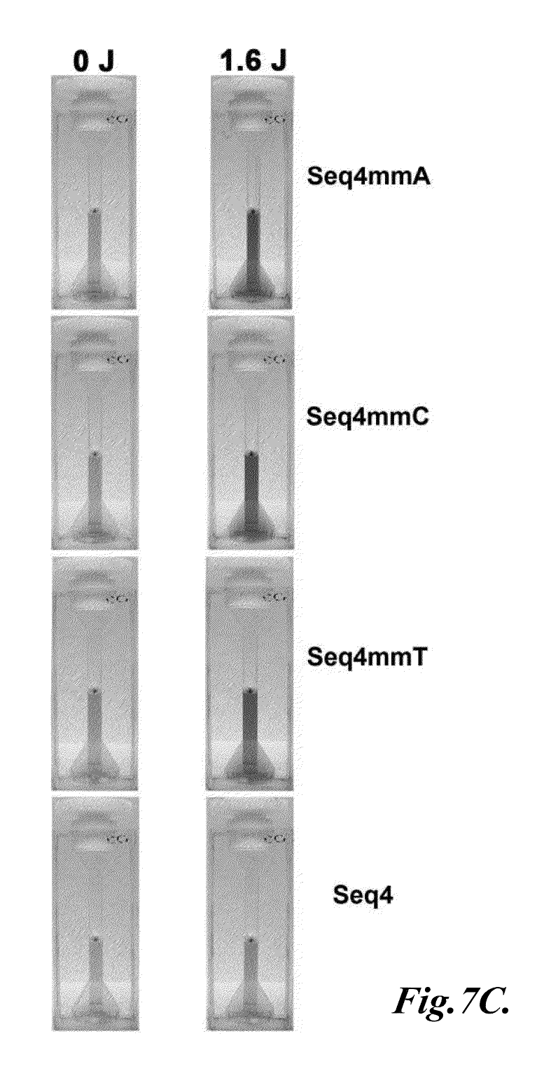

The photoswitchability can be used for the detection of base-pair mismatches. The melting temperature of mismatched DNA, for example, is lower than complementary DNA. This difference is further amplified using the disclosed photoswitchable systems by the cooperative melting of DNA the destabilization from the photoswitchable molecule. For example, the melting temperatures of azoDNA-AuNP with perfect and one base-pair mismatched linkers are ˜60 and ˜37° C. respectively. The rate of photoswitching at 30° C. is therefore expected to be significantly higher for aggregates cross-linked with a mismatched sequence than the perfect complementary DNA. Indeed, after UV irradiation of 30 minutes, the solution of the mismatched linker becomes red due to SPR of single nanoparticles, while the solution of perfectly matched linker remains the same as shown in FIG. 7C.

In at least one aspect, the methods and compositions disclosed herein enable the detection of single base-pair mismatch using light as the probe, as mismatched DNA dehybridizes faster at a given temperature than perfectly matched DNA. The photoswitchable plasmonic property is reversible and can be cycled many times to yield light modulated scattering and absorption signals. Because designed DNA can be used to detect various types of analytes, such as proteins, ions and small molecules, the modulation in optical signal upon binding of the analyte presents a unique sensing platform with broad applications especially in standoff detection.

In sensing applications, the invention allows hybridization stringency between perfect and mismatched sequences to be achieved by controlled photon dose, or controlled photon dose in conjunction with conventional thermal or saline wash conditions.

In another embodiment, the photoswitchability of the system depends on temperature and/or ion (e.g., salt) concentration. In one embodiment, a temperature and an ionic concentration of the solution does not change during said step of altering the hybridization property.

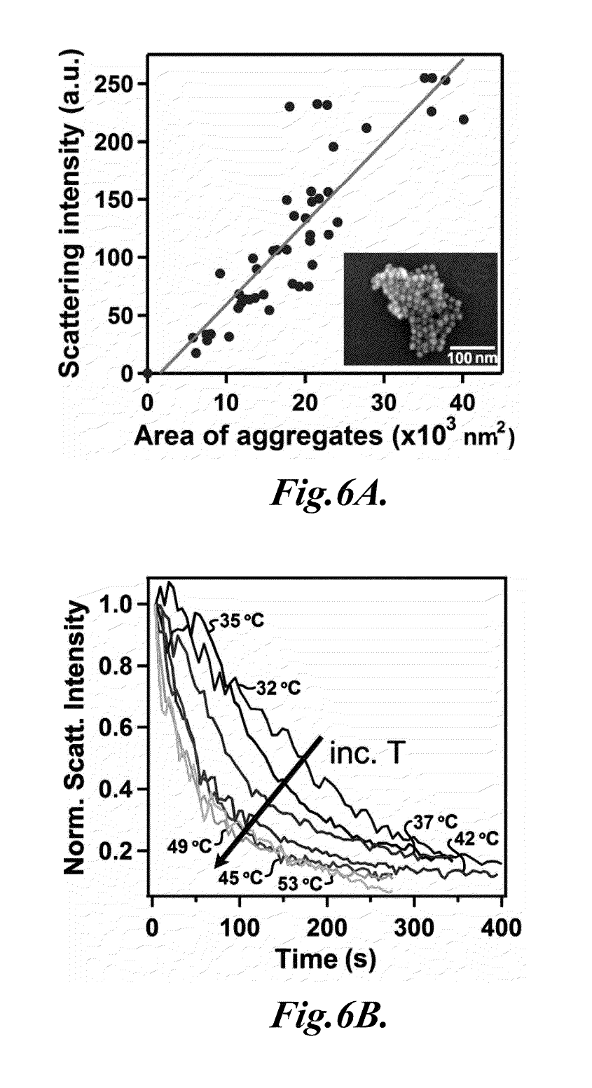

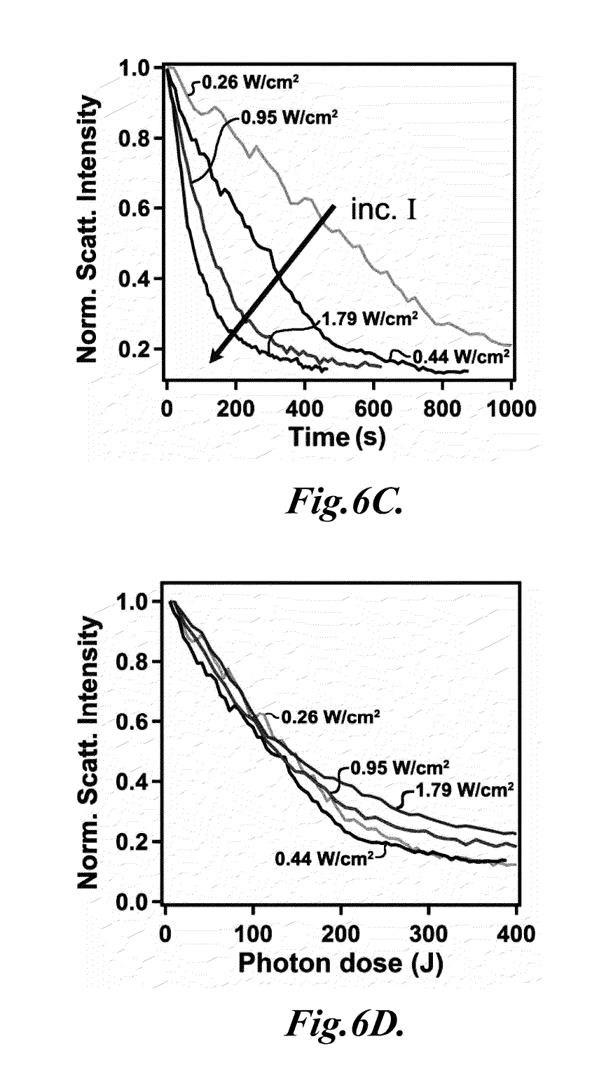

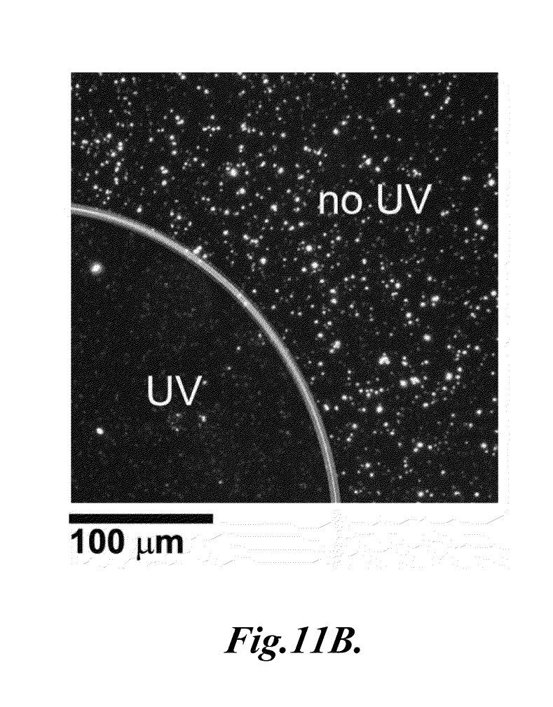

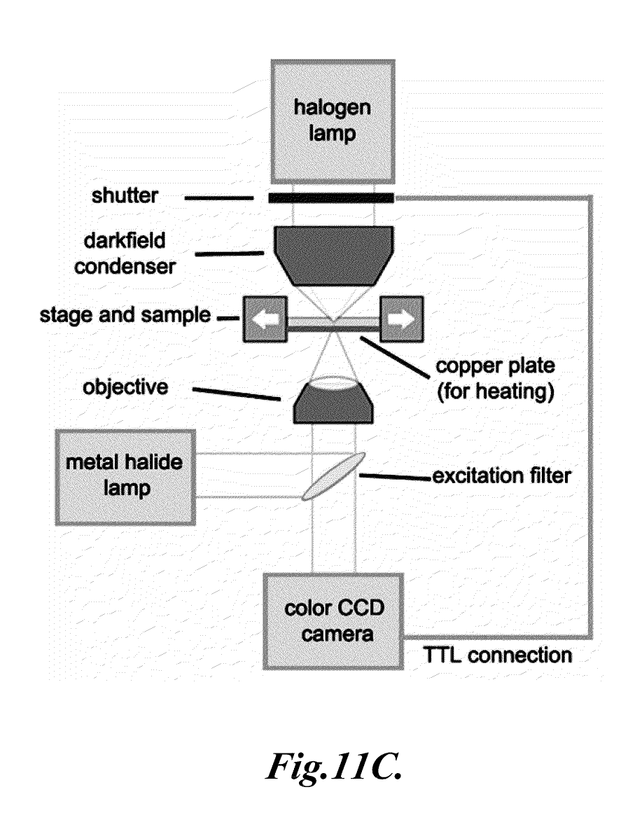

In another embodiment, the photoswitch-modified nucleic acids can be used in chip-based assays. For example, FIG. 11A shows a series of darkfield images of gold-core DNA aggregates formed using azobenzene photoswitching (azoDNA-AuNP) as described elsewhere herein. The sample was irradiated with continuous UV light (with intermittent exposure to darkfield light when images were captured). The aggregates disassemble and the scattering intensity decreases with time. After analyzing 30-40 aggregates and obtaining their average disaggregation kinetics for each temperature (FIG. 6B), it was determined that the rate of photodisaggregation increases with increasing temperature and is independent of photon dose when different intensities of light were employed (FIG. 6D).

The following example is included for the purpose of illustrating, not limiting, the described embodiments.

### EXAMPLE

Gold nanoparticles heavily functionalized with oligonucleotides are widely studied for their unique properties. Gold nanoparticles exhibit localized surface plasmon resonances (LSPRs) that are sensitive to nanoparticle's size and shape, refractive index and interparticle coupling. DNA functionalization of these particles has enabled the programmable assembly of complex nanostructures ranging from plasmonic molecules to 3-D crystals. The unique optical, self-assembly, and biorecognition properties of these particles have been used in biological sensing, chemical sensing, and gene regulation applications.

Generally however, the assembly and binding properties of DNA-functionalized nanoparticles have been controlled primarily by chemical recognition events—e.g., the presence of complementary DNA sequencesor aptamer targets—or by classical inputs that affect DNA hybridization such as salt concentration or temperature. Conferring DNA-nanoparticle conjugates with additional stimulus-response behavior could open many opportunities for new diagnostic, sensing, and nanofabrication applications by enabling the reversible triggering of DNA-directed nanoparticle assembly and associated optical responses.

In this example, we seek to confer such stimulus-response behavior to these versatile materials by functionalizing gold nanoparticles with photoswitchable oligonucleotides. Asanuma and coworkers have shown that trans-azobenzene incorporated into the DNA backbone via a D-threoninol linker will intercalate between natural base pairs in a DNA double strand, raising the melting temperature of the resulting duplex. Upon UV irradiation, trans-azobenzene photoisomerizes to cis-azobenzene thereby destabilizing the DNA duplex. Blue irradiation will photoisomerize the cis-azobenzene back to trans-azobenzene, allowing the modified DNA to rehybridize. By incorporating multiple azobenzene moieties into an oligonucleotide during solid phase synthesis, the hybridization of the resulting DNA duplex can thus be controlled optically.

Here, we show that nanoparticles functionalized with azobenzene-modified photoswitchable oligonucleotides cross-link to form aggregates that can be dissociated to single nanoparticles under UV light, and that the aggregates re-form under blue light. We show that the wavelength-dependent photoisomerization enables remote optical stimuli to modulate the nanoparticle assembly process and therefore control the optical properties of the resulting solution. We further demonstrate a new and useful property of these particles: because the kinetics of the reversible photo-dissociation process depends on temperature and the relative stability of the duplex (i.e., the complementarity of the strands), light can be used to distinguish perfect from partially mismatched targets in hybridization stringency “washes” based on “photomelting.”

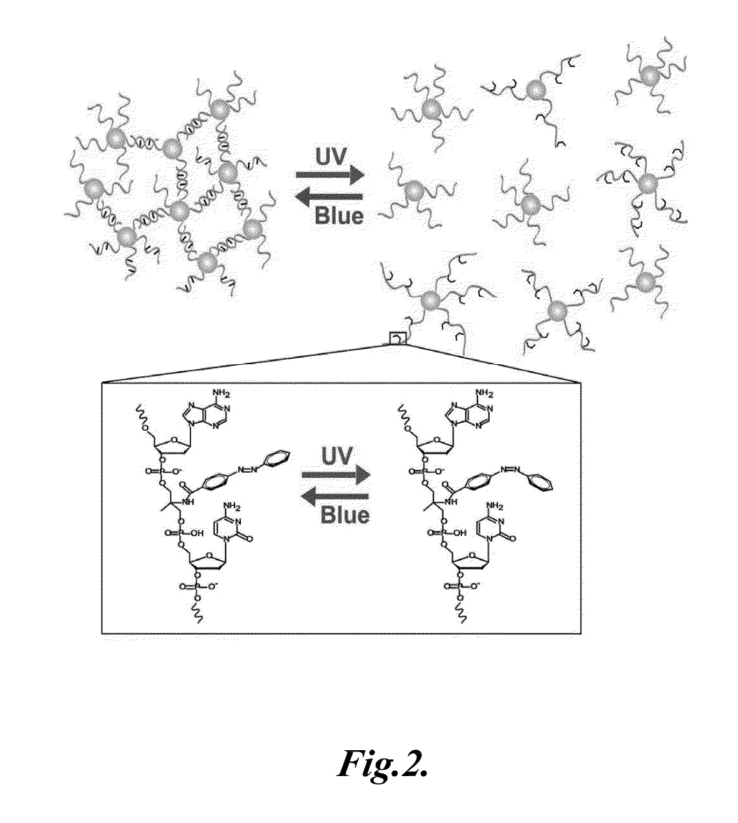

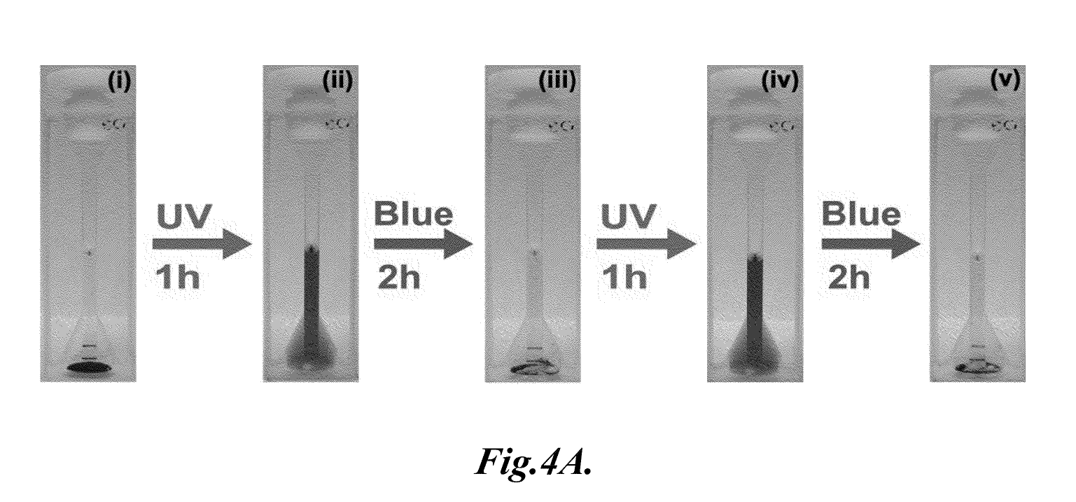

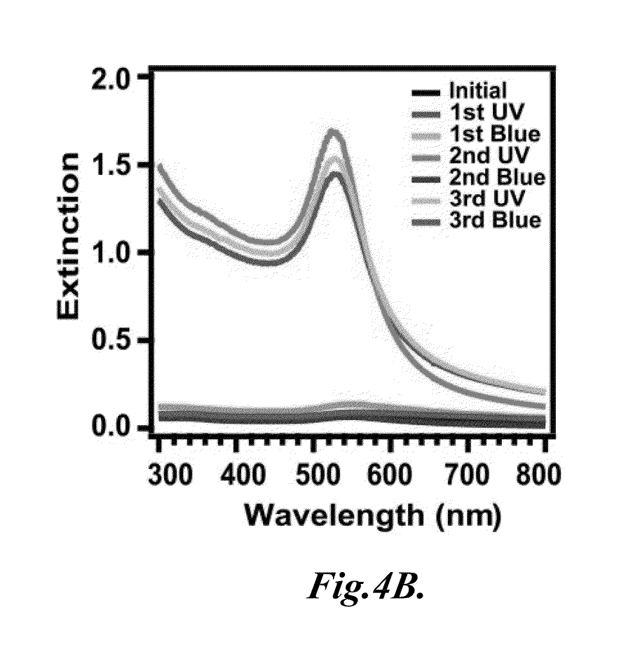

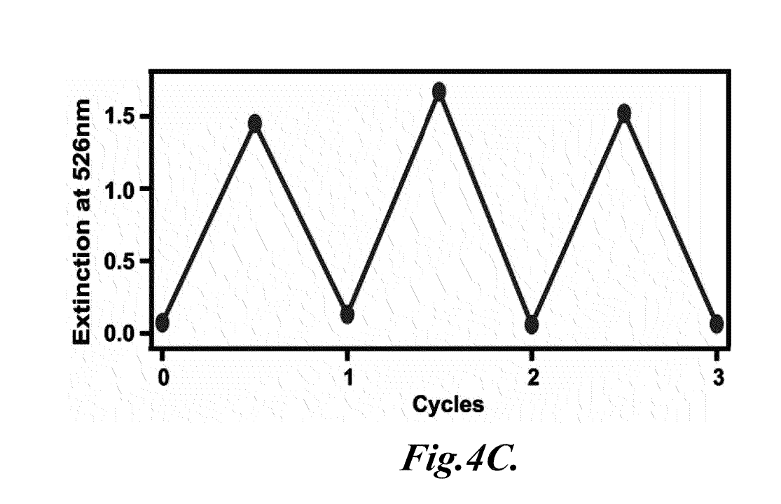

FIG. 2 depicts our approach to obtain photoswitchable DNA-functionalized nanoparticle assemblies. We functionalized one set of nanoparticles with 5′-thiolated azobenzene-modified DNA ([SEQ ID NO:1]) and another with a complementary native 5′-thiolated DNA ([SEQ ID NO:3]) following literature methods for attachment of oligonucleotides to gold nanoparticles and modified as described in supporting information. For our initial experiments, we used a DNA sequence ([SEQ ID NO:1], see FIG. 2), consisting of 10 native bases and 4 evenly spaced azobenzenes, that has been shown to photoswitch reliably in the absence of gold nanoparticles. Once prepared, the resulting DNA-functionalized gold nanoparticle (AuNP) conjugates (denoted as [SEQ ID NO:1]-AuNP and [SEQ ID NO:3]-AuNP) exhibit the classic sequence-specific cross-linking typical of DNA-functionalized gold particles as seen in FIGS. 4A-4C.

FIG. 4A shows a series of photographs demonstrating reversible optical control of DNA-directed nanoparticle assembly with this approach. FIG. 4A(i) shows a solution of a mixture of [SEQ ID NO:1]-AuNPs and [SEQ ID NO:3]-AuNPs (in 0.01 M phosphate buffer, 0.1 M NaCl and 0.01% SDS) held at room temperature for ≧4 h after initial mixing. The solution is nearly colorless because the DNA-linked nanoparticle aggregates have precipitated. The aggregates are visible to the naked eye as black powder on the bottom of the cuvette.

FIG. 4A(ii) shows the same solution after being stirred under UV exposure of 0.83 mW/cm² (UV LED centered at 330 nm, FWHM≦10 nm) for 1 h at 45° C. (15° C. below gold nanoparticle assemblies' melting temperature). The solution is the bright red characteristic of dispersed gold nanoparticles (15 nm in diameter) as a result of the UV-induced photomelting of the double-stranded DNA linking the aggregates together.

Exposure to blue light reverses the process, allowing the nanoparticles to reassemble into large aggregates. FIG. 4A(iii) shows a photograph of the same solution after turning off the UV light and further exposure of the solution to 11 mW/cm² of blue light from the LED (wavelength centered at 470 nm, FWHM of 30 nm). After 2 h of blue irradiation under stirring and additional 20 min in the dark without stirring (to allow complete precipitation), the solution has again become colorless, with the nanoparticle aggregates visible to the naked eye as fine black powder on the bottom of the cuvette. FIGS. 4A(iv) and 4A(v) display images of the same solution after one more cycle of UV and blue illumination with the same experimental treatment.

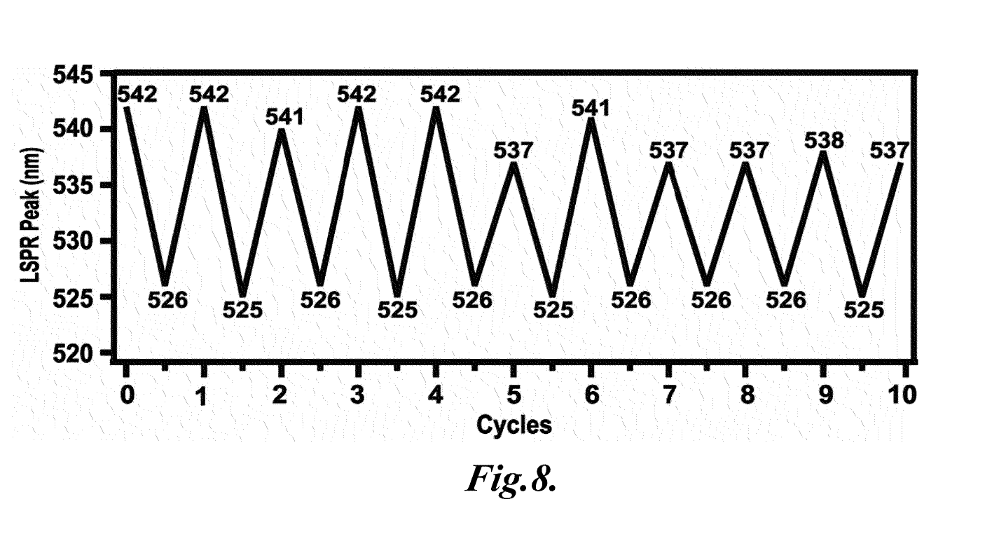

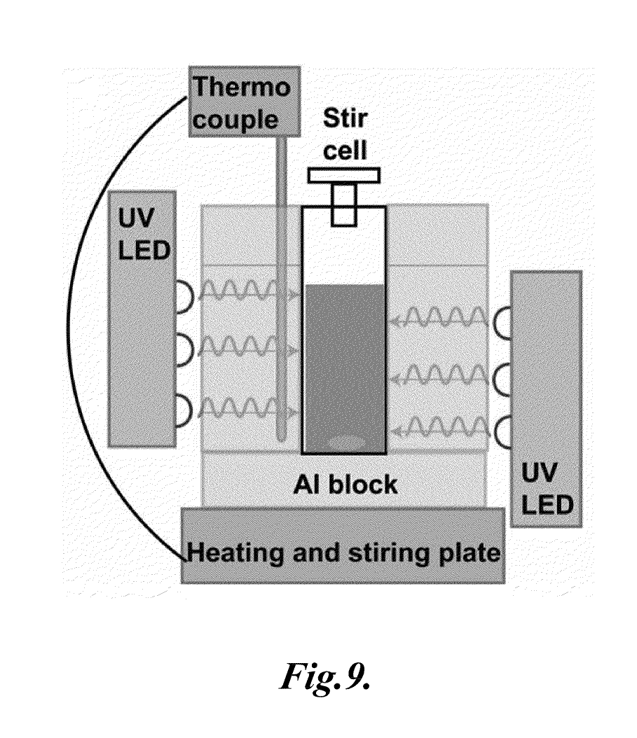

As anticipated, the photoisomerization process is reversible over many cycles. FIGS. 4B and 4C show the UV-vis extinction spectra and extinction changes at 526 nm, respectively, for the same solution cycled 3 times between the completely disaggregated and sedimented states. One hour of UV irradiation leads to an increase of the solution's extinction to around 1.5, with narrow LSPR at 526 nm, as large aggregates dissociate and the single gold nanoparticles become resuspended. Then, after 2 h of blue irradiation the nanoparticles again fully precipitate as large aggregates and the solution exhibits an extinction of almost zero. The negligible variations in the extinction change after each cycle confirm that we can achieve complete and reversible photoswitching with these nanoparticles. (Additional data on reversible photoswitching for more cycles can be found in FIG. 8.)

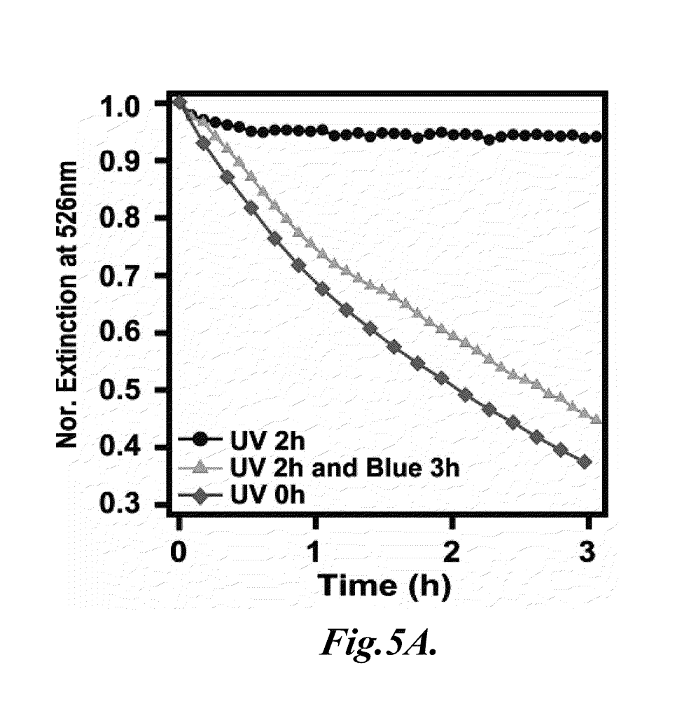

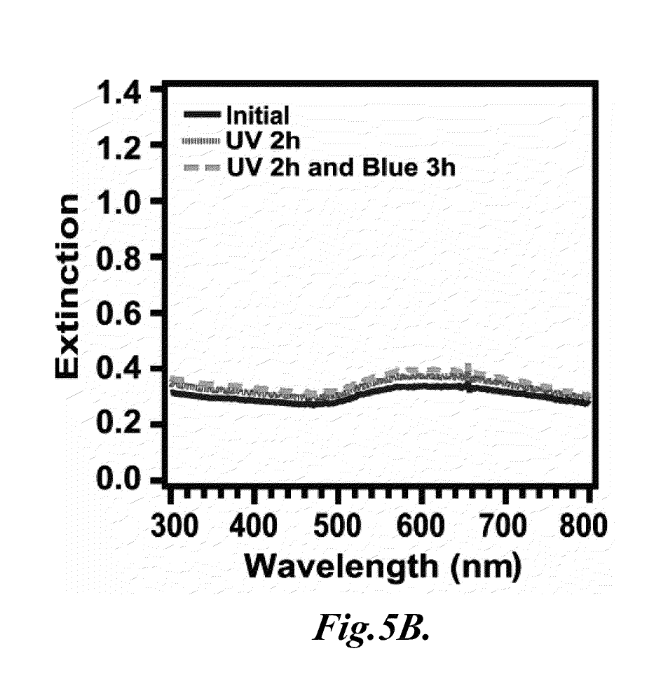

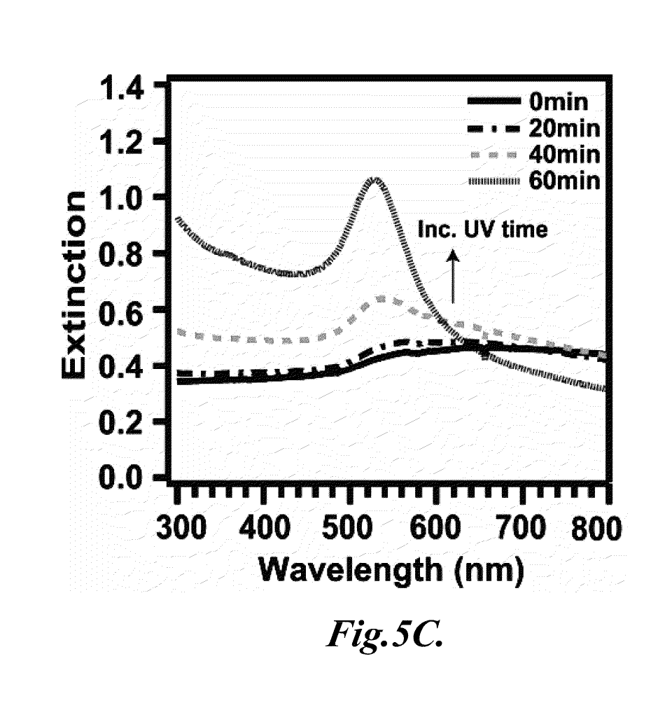

While gold nanoparticles can undergo local heating when illuminated, and unusual release of DNA from gold nanoparticles under laser exposure has been reported, we are confident that the photomelting we describe here is due to the robust photoisomerization of the azobenzene modifications. First, the relative intensities of the UV and blue LEDs used to collect the data in FIG. 1 are such that the blue LEDs deliver at least 10 times more absorbed power to the sample than the UV LEDs (see supporting information). Nevertheless the blue LEDs cause hybridization and aggregation, while the lower powered UV LEDs cause the nanoparticle assemblies to dissociate—as expected for photoisomerization-controlled melting, but inconsistent with reported photothermal and light-induced DNA release mechanisms. As an additional control experiment, we irradiated the [SEQ ID NO:1]-AuNPs with UV light prior to mixing them with the [SEQ ID NO:3]-AuNPs. The resulting cis-form [SEQ ID NO:1]-AuNPs show very little aggregation with the [SEQ ID NO:3]-AuNPs, even hours after the illumination when no residual local heating or light-induced melting could possibly be present (FIG. 5A). In contrast, an identically prepared control mixture without any pre-mixing illumination show fast aggregation, as does a solution of [SEQ ID NO:1]-AuNPs that was exposed to blue light (to photoisomerize the cis-azobenzene back to trans-azobenzene) immediately after UV exposure. Furthermore, we irradiated gold nanoparticle aggregates linked by DNA without azobenzene modification ([SEQ ID NO:2] and [SEQ ID NO:3]) with UV and blue light as a control, and we observed no change in the extinction spectra (FIG. 5B). This observation is in stark contrast to the spectral evolution under UV light for aggregates linked by azobenzene-modified DNA ([SEQ ID NO:1]-AuNPs and [SEQ ID NO:3]-AuNPs, FIG. 5C), where the solution extinction gradually increases and LSPR sharpens and blue shifts eventually to 526 nm, matching the spectrum of individually dispersed 15 nm-diameter gold nanoparticles.

We note that while azobenzene-based photocontrol of gold nanoparticle aggregation has been reported previously, these earlier demonstrations used azobenzene that was directly covalently bonded to the gold nanoparticles using alkanethiol linkages. The advantage of our approach is that it combines the opportunities of photoswitch-based control with the programmable recognition properties of DNA-functionalized nanoparticles to enable new applications. For example, aside from the obvious applications in light-controlled DNA-programmed nanoscale assembly, photoswitchable DNA-nanoparticle conjugates could be useful to discriminate specific binding from nonspecific target interference in diagnostics: because foreign or interfering species can often cause nanoparticles to precipitate from solution or adhere to a substrate (resulting in false positives) in colorimetric assays, we suggest that photomelting of DNA-linked aggregates could be used as a general strategy to confirm the presence of specific targets. These photoswitchable DNA-nanoparticle conjugates also enable a unique new form of DNA-hybridization stringency as we demonstrate below.

In order to better understand the photomelting process, we investigated the temperature and photon dose dependence of the light-induced disaggregation process. Because the relationship between the ensemble solution extinction spectrum and aggregate size is complicated by the heterogeneity and precipitation of the large aggregates, we used darkfield microscopy to measure the kinetics of photomelting on many individual, surface-attached aggregates in parallel. FIG. 6A shows SEM calibration data confirming that the light-scattering intensity scales linearly with aggregate area. There is more spread in our data for larger aggregates because they can be multilayer and have a wider distribution in the number of particles, but the linear correlation holds well for smaller aggregates. Hence we can derive the disaggregation kinetics by analyzing the temporal evolution of the scattered light intensity from a series of darkfield images (FIG. 11A) by choosing aggregates that fall on the linear calibration curve. In a typical experiment, the sample was irradiated with continuous UV light at 375 nm, with different intensity shown in FIG. 6B, for a total of 8-16 min with intermittent brief 106 ms exposure to darkfield light ranging from 2 to 6 s when images were captured. As the aggregates disassemble, the scattering intensity decreases with time. Typically, we analyzed 30-40 individual aggregates to obtain average disaggregation kinetics under a given set of conditions.

FIG. 6B shows the disaggregation kinetics at a series of temperatures from 32° C. to 53° C. for nanoparticle aggregates linked by [SEQ ID NO:1] and [SEQ ID NO:3]. Under these conditions, the DNA-linked nanoparticle aggregates photomelt following ˜6 min of exposure to UV light. It is clear that, even well below the melting temperature, the photoinduced disaggregation is temperature dependent and becomes faster at higher temperatures. We speculate that this temperature dependence may be associated with the degree of local thermal motion in the DNA helix surrounding the azobenzene photoswitches that ultimately influences the rate of photoisomerization-induced dehybridization.

FIG. 6C shows that the light-scattering intensity of the anchored gold nanoparticle assemblies drops faster with higher UV intensity. The photomelting rate thus depends on the intensity of the UV illumination, suggesting that faster photomelting could be achieved using sufficiently intense light sources. Importantly, over the intensity ranges and temperatures investigated here, photomelting appears to be controlled by total UV photon dose. FIG. 6D shows that the measured dissociation curves overlap when the x-axis is plotted as total photon dose, even though melting likely requires absorption of multiple photons since there are multiple azobenzenes per DNA strand, and multiple DNA strands per nanoparticle. Such photon dose dependent behavior is consistent with a negligible rate of cis-trans thermal isomerization over the course of the experiment. This dose-dependent behavior could be used, for example in microfluidic and lab-on-a-chip application to minimize the need for heating, mixing and fluid processing—thus reducing system complexity.

Since the kinetics of photoinduced disaggregation depends on temperature, we hypothesized that it would be possible to distinguish mismatched sequences from perfect sequences via controlled photon dose during photomelting to achieve a “photon stringency wash.” A stringency wash typically involves washing with buffers of different temperatures, or ionic strength, with the temperature/ionic strength chosen so that the perfect complement (which is thermodynamically more stable) remains bound to the probe DNA, while the mismatched targets are preferentially dehybridized. Distinguishing between perfect complements and partial mismatches is an important part of DNA and RNA assays. Next, we demonstrate that photon dose stringency can indeed be used instead of conventional temperature- or salt-dependent stringency washes to distinguish single-base mismatches in target strands using these new photoswitchable DNA-functionalized nanoparticles.

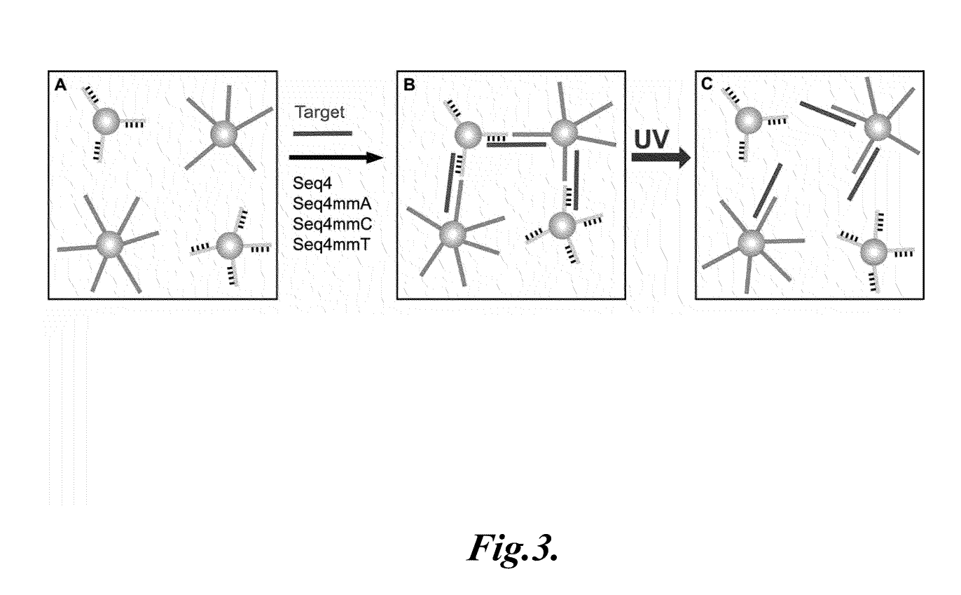

For these experiments, we utilized a classic 3-strand target-probe capture strategy as depicted in FIG. 3: gold nanoparticles are functionalized with [SEQ ID NO:1] and [SEQ ID NO:4], which are each complementary to opposite ends of the target sequence ([SEQ ID NO:5]). When mixed, solutions of gold nanoparticles that are functionalized with [SEQ ID NO:1] and [SEQ ID NO:4] thus form aggregates in the presence of the [SEQ ID NO:5] target. In addition to the perfectly complementary target, nearly complementary targets with partial sequence mismatches can also cause cross-linking of the nanoparticles. Although the target linking strand is native DNA, [SEQ ID NO:1] (attached to the gold nanoparticles) contains 4 azobenzenes and the resulting nanoparticle aggregates can thus be reversibly photoswitched (FIG. 12).

FIGS. 7A-7C shows that the photoswitchable gold nanoparticles allow photon dose to be used to achieve hybridization stringency and discrimination of single base mismatches. FIGS. 7A and 7B show the photomelting data for four solutions of nanoparticles, one with a perfectly complementary target ([SEQ ID NO:5]) and three with single-base mismatches ([SEQ ID NO:6], [SEQ ID NO:7], [SEQ ID NO:8], see FIG. 3) during exposure to 330 nm light from UV LEDs at 30° C. We track the photomelting process by monitoring the UV-vis spectra of the solutions and plotting the LSPR peak (FIG. 7A) and extinction change (FIG. 7B) as a function of photon dose. FIG. 7A shows that mismatch-linked assemblies photomelt to almost all single nanoparticles with LSPR peaks shifting from ˜560 nm to 526-528 nm. However, assemblies linked by the complementary target remain as small aggregates with a very small LSPR shift from 566 nm to 557 nm. We further examine the extinction changes at 526 nm because dispersed single gold nanoparticles in our experiment have the strongest optical response at that wavelength. FIG. 7B shows that gold nanoparticle assemblies linked by [SEQ ID NO:7] and [SEQ ID NO:6] exhibit the fastest rise in extinction as they photomelt, with an overall change in extinction of ˜0.5 after 1.6 J of UV light exposure. Nanoparticles linked by [SEQ ID NO:8] also photomelt, though to a lesser degree over the same time period (extinction change of 0.3). On the other hand, the complementary target linked gold assemblies yield only a small increase in extinction. This trend in the photomelting kinetics is in line with the relative stability of the aggregates (i.e., the melting temperatures of assemblies are [SEQ ID NO:7]≈[SEQ ID NO:6]<[SEQ ID NO:8]<<[SEQ ID NO:5], see Table 1). These changes in extinction can also be visualized by the color change of the solution as shown in the photographs of FIG. 7C. Initially, after stirring and equilibration in the dark, the solutions are faintly pink as the aggregates are uniformly suspended inside the cell (not sedimented). After 1.6 J photon dose of UV irradiation, the color of mismatched samples transitions to red due to photomelting of the DNA-linked gold nanoparticle aggregates. In contrast, the extinction of perfect target solution hardly changes—indicating little photomelting of gold nanoparticle aggregates. These colorimetric changes demonstrate that facile discrimination of mismatches can be achieved using light, and that more detailed analysis could possibly further differentiate between the types of mismatches under certain circumstances.

TABLE 1
Melting temperatures (Tₘ) of 3-strand AuNP assemblies at
0.01M phosphate buffer, 0.05M NaCl and 0.01% SDS.
The temperature ramp starts at 20° C. and ends at 60° C.
or 80° C. with 1° C. temperature steps and 2 min hold time.

| AuNP assemblies | Tₘ (° C. ) |
|---|---|
| [SEQ ID NO: 5] | 60.5 |
| [SEQ ID NO: 6] | 40.5 |
| [SEQ ID NO: 7] | 41.5 |
| [SEQ ID NO: 8] | 44.9 |

Although it is still early to predict the full utility of the photostringency condition, we suggest that it might be preferred for the following reasons: (1) light intensity can be controlled more readily than temperature, pH or ionic strength; (2) photomelting can be accelerated at higher intensity, so the stringency wash could potentially be faster using more intense illumination (indeed we achieved full dissociation in minutes in the optical microscope geometry); (3) photostringency could reduce the complexity of microfluidic systems such as heaters/mixers and valves in lab-on-a-chip hybridization applications; (4) photostringency enables remote manipulation without contacting the sample; (5) reversibility could provide a chance to recover DNA after the stringency “wash.”

In summary, we have prepared and characterized photoswitch-modified DNA-nanoparticle conjugates. These particles combine the biological functionality and programmable assembly properties of conventional DNA-nanoparticle conjugates with the reversible stimulus-responsive properties of photoswitch-modified nano-materials. In addition to light-triggered self-assembly applications, we anticipate these nanoparticles could have immediate applications in reducing false positives in colorimetric assays, and further offer the potential to establish new sensing platforms by speeding up analysis, reducing the complexity of microfluidic devices for DNA hybridization assays, or by enabling interrogation of remote standoff sensors via a pump-and-probe methodology. Importantly, we demonstrate that these materials provide a unique new means for distinguishing base-pair mismatches in DNA targets via photostringency. Given the ubiquity of DNA assays, the wide range of biological molecules that can be designed for specific targets, and the readily available capability of incorporating photoactive molecules, we envision these systems will find wide application.

### Preparation of DNA Functionalized Gold Nanoparticle Aggregates

Colloidal gold nanoparticles (AuNPs) of 15 nm in diameter (˜10¹² particles/mL) were purchased from Ted Pella Inc. and used as received. All oligonucleotides were purchased from Integrated DNA Technology. Water used was deionized to 18.2 MOhm with Millipore filtration system.

(i) Functionalizing AuNPs with DNA

Aliquots of thiolated DNA sequences ([SEQ ID NO:1], [SEQ ID NO:2], [SEQ ID NO:3], [SEQ ID NO:4]) were freshly cleaved by incubating with 0.1 M DTT (dithiothreitol, 0.17 M phosphate, pH=8.0) for 15 min. and subsequently purified with a Bio-Spin Column. The resulting volume of 30-40 μL of DNA in water (O.D.=˜2.0 at 260 nm) was combined with 900 μL of 15 nm AuNP solution. The mixture was vortexed and sonicated, and left for 20 min at room temperature. The solution was then brought to 0.01% SDS using 1% SDS stock and 0.01 M PBS (Phosphate buffered saline, 0.137 M NaCl) using 0.1 M PBS stock. In the subsequent steps, the NaCl concentration was gradually raised to 0.4 M in increments of 0.05-0.1 M by adding buffer solution of 2 M NaCl, 0.01 M PBS and 0.01% SDS while keeping the concentration of PBS and SDS constant. After each addition of NaCl, the solution was sonicated for 1 min. and then incubated for 20 minutes (at room temperature for most sequences, 45° C. for [SEQ ID NO:1]-AuNP) and finally incubated overnight at 0.4 M NaCl at room temperature. The DNA-functionalized AuNP (DNA-AuNP) samples were washed 3 times by repeated centrifugation and redispersion into 400 μL 0.01% SDS solution, and eventually diluted back to 900 μL of 0.01 M phosphate buffer (pH=6.6), 0.1 M NaCl, 0.01% SDS and 0.02% sodium azide for storage at 4° C. in the dark.

DNA Sequences:

[SEQ ID No: 1]
5′-S-AAAAAAAAATGNAANCTNAANCG-3,′ N = Azobenzene;
[SEQ ID No: 2]
5′-S-AAAAAAAAATGAACTAACG-3′;
[SEQ ID No: 3]
5′-S-AAAAAAAAAACGTTAGTTCA-3′;
[SEQ ID No: 4]
5′-CAATCATGAGCAGCCTAGCAGAGAAGTAAAAAAAAAA-S-3′;
[SEQ ID No: 5]
5′-ACTTCTCTGCTAGGCTGCTCATGATTGCGTTAGTTCA-3′;
[SEQ ID No: 6]
5′-ACTTCTCTGCTAGGCTGCTCATGATTGCGTTAATTCA-3′;
[SEQ ID No: 7]
5′-ACTTCTCTGCTAGGCTGCTCATGATTGCGTTACTTCA-3′;
and
[SEQ ID No: 8]
5′-ACTTCTCTGCTAGGCTGCTCATGATTGCGTTATTTCA-3′

(ii) Preparing Colloidal Aggregates for Photoswitching Experiments

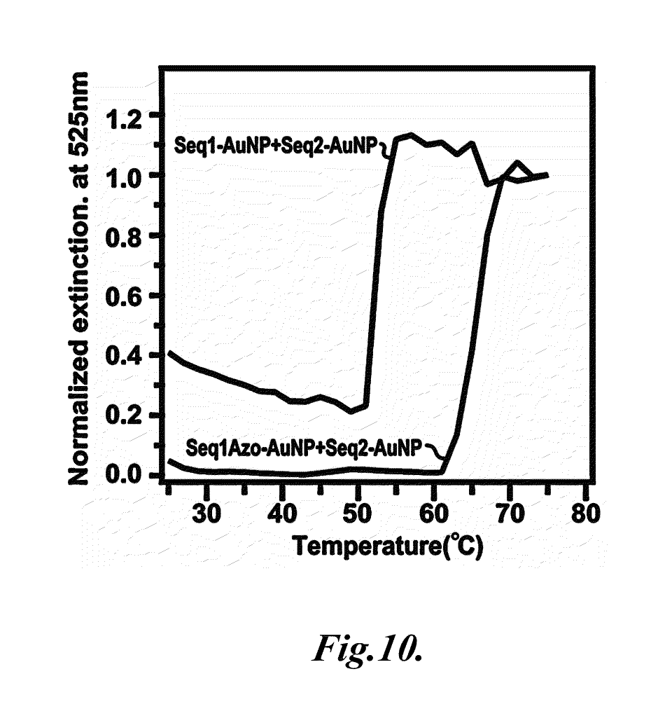

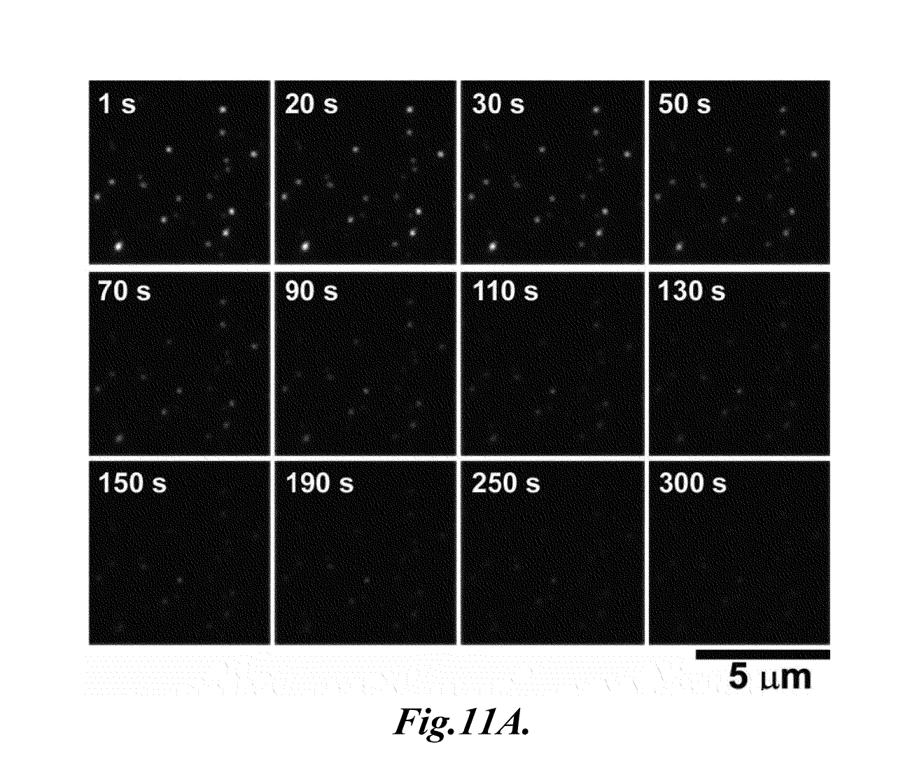

For a typical photoswitching experiment, 600 μL of solution containing azobenzene-modified DNA-AuNP aggregates was placed inside a stirring cuvette (Starna Cells). The aggregates were formed by mixing equal amounts of [SEQ ID NO:1]-AuNPs and [SEQ ID NO:3]-AuNPs for at least 4 h at room temperature. For the 3-strand motif, a volume of 300 μL of [SEQ ID NO:4]-AuNP solution was first combined with 3 μL 10-5 M [SEQ ID NO:5] linker (or [SEQ ID NO:6], [SEQ ID NO:7], [SEQ ID NO:8]), annealed at 45-50° C., and then incubated at 25° C. for ≧4 h followed by the addition of 300 μL of [SEQ ID NO:1]-AuNPs at room temperature. The melting temperature for [SEQ ID NO:1]-AuNP +[SEQ ID NO:3]-AuNP is 61.7° C. and that of [SEQ ID NO:2]-AuNP +[SEQ ID NO:3]-AuNP is 52.1° C. (in 0.01 M PBS, 0.1 M NaCl and 0.01% SDS, see FIG. 10). Values were determined with extinction-based melting curves using the Agilent 8453 UV-Vis spectrometer, and Tm points were obtained with its built-in denaturalization mode. The temperature ramp starts at 25° C. and ends at 75° C. with 1° C. temperature steps and 2 min hold time.

### Photoswitching Azobenzene-Modified DNA-AuNP Aggregates in Solution

The photoswitching setup consists of an LED light source, an aluminum block (with light accessible windows) as a thermal mass holding the quartz cuvette, and a temperature-controlled stirring plate with a thermocouple inserted into the Al block. It was verified that the cuvette solution temperature was the same as the thermocouple setpoint. The UV LEDs (UVTOP325HS) were purchased from Sensor Electronic Technology, Inc., and blue LEDs (Rebel 7, 470 nm) fitted with Optics Lens (Polymer Optics 264 7 Cell Cluster 12 Diffused Optic® array) were obtained from LuxeonStar®. Typically, 4 UV LEDs at ˜7 cm away from the cuvette were aligned to give a total power of 0.83 mW/cm², as measured by a calibrated Si photodiode. Blue LEDs gave uniform illumination at 11 mW/cm².

Prior to any irradiation, the sample was thermally equilibrated for at least 15 minutes. For [SEQ ID NO:1]-AuNPs and [SEQ ID NO:3]-AuNPs in 0.01 M PBS, 0.1 M NaCl and 0.01% SDS, photoswitching was performed at 45° C. For discriminating between different [SEQ ID NO:5] linkers, photostringency was performed at 30° C. in 0.01 M PBS, 0.05 M NaCl and 0.01% SDS. UV-vis spectra of the solution were taken with an Agilent 8453 diode array spectrophotometer.

### Photomelting Surface-Anchored Azobenzene-Modified DNA-AuNP Aggregates

To attach individual aggregates on a substrate, a drop of 10 μL solution containing [SEQ ID NO:1]-AuNP and [SEQ ID NO:3]-AuNP aggregates was place on top of a cover slip glass slide that had been silanized with 3-aminopropyltrimethoxysilane. After a few minutes, the substrate was rinsed with a solution of 0.01 M phosphate buffer and 0.1% SDS, then with 0.3 M ammonium acetate and dried with nitrogen stream. The sample filled with 0.01 M phosphate buffer, 0.05 M NaCl in a SecureSeal hybridization chamber (Grace Biolab) was mounted on a homemade copper heating stage with a temperature controller. The temperature difference between the sample and the copper block was corrected via a calibration curve. Darkfield scattering images were captured using a thermoelectrically cooled color CCD camera (Diagnostic Instruments, FX1520) coupled to a Nikon TE-2000 inverted microscope fitted with a transmitted dark-field condenser and a 50× objective (Nikon Plan RT, NA 0.7, CC 0-1.2) with an intermediate 1.5× lens (total magnification 75×). A standard tungsten halogen lamp was used for transmitted dark-field illumination, and metal halide lamp (EXFO X-Cite 120) with a 350 nm±40 nm filter was used as UV light source. The resulting UV light (after passing through microscope optics) showed a peak wavelength at 375 nm. A homemade shutter triggered by the TTL output of the camera blocked the darkfield halogen lamp illumination except during image capture to prevent interference with the kinetics from the visible light in the darkfield illumination. All scattering images were analyzed using Igor Pro software. Correlated scanning electron microscope (SEM) images of the aggregates for the calibration curve were obtained using FEI Sirion (University of Washington NanoTech User Facility), with the area of aggregate extrapolated using ImageJ software.

### Discussions of Photothermal Effect

We calculate the maximum energy from UV and blue LEDs absorbed by single AuNPs. Note that the extinction of the aggregated solutions is actually lower than for the dispersed single AuNPs; hence the value calculated here is an overestimate. From FIG. 4B, dispersed single AuNPs exhibit an extinction of 1.14 at wavelength 330 nm and 0.978 at wavelength 470 nm, corresponding to absorption of 93% of the UV and 90% of the blue light, respectively (scattering is negligible for 15 nm-diameter AuNP). Based on the amount of absorption, the intensity of the LEDs and the illumination area on the sample (0.601 cm²), the particles absorb 1.7 J of UV light after 1 h and 42 J of blue light after 2 h irradiation under the conditions we employed in our experiments. These energies are substantially lower than what is typically required for photothermal melting.

Additionally the temperature increase on the surface of an individual nanoparticle in aqueous solution has been as

$$Δ T = \frac{{σ}_{abs} I}{4 π {R}_{eq} {βκ}_{water}}$$

σ_{abs} is the absorption cross section (m²), I is the light intensity (W/m²), R_{eq} is the radius of a sphere (m), β is the thermal capacitance coefficient (1 for spherical particles), and κ_{water} is the thermal conductivity of water (0.6 W/m*K). For gold nanoparticles with σ_{abs}˜10⁻¹⁵ m², the temperature changes upon exposure to 330 nm UV light at the power used here are 1.5×10⁻⁷ K, and 1.9×10⁻⁶ K for blue (470 nm) light. The negligible heating strongly argues against photothermal heating as the disassembly mechanism, as confirmed by the native DNA (without azobenzene modification) controls. Furthermore, the more intense blue LED causes hybridization and aggregation in our experiment, while the lower powered UV LED causes melting. Hence the assembly process agrees with photoisomerization-controlled melting mechanism.

While illustrative embodiments have been illustrated and described, it will be appreciated that various changes can be made therein without departing from the spirit and scope of the invention.

The embodiments of the invention in which an exclusive property or privilege is claimed are defined as follows:

1. A composition, comprising:
a surface;
a first nucleic acid sequence attached to the surface; and
a photoswitchable molecule incorporated into the first nucleic acid sequence; wherein the photoswitchable molecule is capable of undergoing a structural change from a first conformation to a second conformation upon illumination by a first wavelength of light, wherein the structural change alters a hybridization property of the first nucleic acid sequence in relation to a second nucleic acid sequence.

2. The composition of claim 1, wherein the first nucleic acid sequence and the second nucleic acid sequence are located on separate nucleic acid strands.

3. The composition of claim 1, wherein the first nucleic acid sequence and the second nucleic acid sequence are located on the same nucleic acid strand.

4. The composition of claim 1, wherein the hybridization property is altered to destabilize hybridization of the first nucleic acid sequence with the second nucleic acid sequence.

5. The composition of claim 1, wherein the hybridization property is altered to stabilize hybridization of the first nucleic acid sequence with the second nucleic acid sequence.

6. The composition of claim 1, wherein the structural change is reversible upon illumination by a second wavelength of light that is different than the first wavelength of light.

7. The composition of claim 6, wherein the first wavelength is less than the second wavelength and the hybridization property is altered to stabilize hybridization of the first nucleic acid sequence with the second nucleic acid sequence.

8. The composition of claim 6, wherein the first wavelength is less than the second wavelength and the hybridization property is altered to destabilize hybridization of the first nucleic acid sequence with the second nucleic acid sequence more likely.

9. The composition of claim 6, wherein the first wavelength is greater than the second wavelength and the hybridization property is altered to destabilize hybridization of the first nucleic acid sequence with the second nucleic acid sequence more likely.

10. The composition of claim 6, wherein the first wavelength is greater than the second wavelength and the hybridization property is altered to stabilize hybridization of the first nucleic acid sequence with the second nucleic acid sequence.

11. The composition of claim 6, wherein the first wavelength and the second wavelength are independently selected from the group consisting of near-infrared, visible, and ultraviolet wavelengths.

12. The composition of claim 1, wherein the photoswitchable molecule is intercalated into the first nucleic acid.

13. The composition of claim 1, wherein the photoswitchable molecule is covalently attached to the first nucleic acid sequence and intercalates between the first nucleic acid sequence and the second nucleic acid sequence when hybridized.

14. The composition of claim 1, wherein the surface is a particle core having a shape selected from the group consisting of a sphere, a cylinder, an ellipsoid, a polyhedron, a prism, a rod, and a wire.

15. The composition of claim 14, wherein the core is optically detectable by changes in absorption, light scattering, or photoluminescence that are triggered by changes in the hybridization state of the first nucleic acid sequence in relation to the second nucleic acid sequence.

16. The composition of claim 14, wherein the core has a surface plasmon resonance.

17. The composition of claim 14, wherein the core has a critical dimension of from 1 nm to 200 nm.

18. The composition of claim 1, wherein the surface is a planar surface on a substrate.

19. The composition of claim 18, wherein the surface is part of an assay chip.

20. A method of altering a hybridization property between a first nucleic acid sequence and a second nucleic acid sequence, the method comprising the steps of:
(a) providing a solution, comprising:
(i) a first nucleic acid sequence attached to a surface; and
(ii) a second nucleic acid sequence that is not attached to the surface; wherein at least one of the first nucleic acid sequence and the second nucleic acid sequence incorporates a photoswitchable molecule; wherein the photoswitchable molecule is capable of undergoing a structural change from a first conformation to a second conformation upon illumination by a first wavelength of light, wherein the structural change alters a hybridization property of the first nucleic acid sequence in relation to the second nucleic acid sequence; and
(b) altering the hybridization property by illuminating the photoswitchable molecule with the first wavelength of light.

21. The method of claim 20, wherein the photoswitchable molecule is incorporated into the first nucleic acid sequence.

22. The method of claim 20, wherein the photoswitchable molecule is incorporated into the second nucleic acid sequence.

23. The method of claim 20, the first nucleic acid sequence and the second nucleic acid sequence are complementary.

24. The method of claim 20, wherein the first nucleic acid sequence and the second nucleic acid sequence are partially complementary and partially non-complementary when hybridized.

25. The method of claim 24, wherein the partially non-complementary first nucleic acid sequence and second nucleic acid sequence results from a non-complementary arrangement selected from the group consisting of a nucleic acid mismatch, an abasic site, a modified base, and combinations thereof.

26. The method of claim 24, wherein the structural change destabilizes hybridization of the first nucleic acid sequence with the second nucleic acid sequence, and wherein said destabilized hybridization requires a first amount of photonic energy that is less than a second amount of photonic energy as defined by the amount of photonic energy required to destabilized hybridization of the first nucleic acid with the second nucleic acid to the same extent if they were more complementary.

27. The method of claim 26, wherein a temperature and an ionic concentration of the solution does not change during said step of altering the hybridization property.

28. The method of claim 20, wherein the structural change is reversible upon illumination by a second wavelength of light that is different than the first wavelength of light.

29. The composition of claim 1, wherein the surface is a particle core having a shape selected from the group consisting of a sphere, a cylinder, an ellipsoid, a polyhedron, a prism, a rod, and a wire.
